(19) United States
(12) Patent Application Publication
(10) Pub. No.: US 2010/0122419 A1
(43) Pub. Date: May 20, 2010

(54) Multi-Function Tool for Healthcare Professionals

(76) Inventor: Amber Zupancic-Albin, Lake Oswego, OR (US)

Correspondence Address: Layne J. McWilliams, PE, Esq., 5335 Meadows Road, Suite 161, Lake Oswego, OR, 97035, US

(21) Appl. No.: 12/271,351

(22) Filed: Nov. 14, 2008

(51) Int. Cl. B25B 7/22; A61B 17/00

(52) U.S. Cl. 7/125; 606/1

(57) ABSTRACT

A multi-function tool designed for the needs of healthcare professionals. The multi-tool may include individual tools that a healthcare professional uses on a regular basis, including a gas cylinder wrench, an integrated pill slicer, a set of EKG calipers, a sharp/dull neurological response tester, and a gripping device for I.V. caps. Additional features may include a penlight or laser pointer and a fold out ruler. A tool carrier includes features that healthcare professionals also regularly use, such as a tape roll holder and dispenser and retractable ring for keys and security badges.

### FIELD OF THE INVENTION

This invention relates to multi-function tools configured with features that are particularly relevant and useful for healthcare professionals, including doctors, nurses, and first responders.

### BACKGROUND OF THE INVENTION

The folding multi-function tool (also called a multipurpose tool or simply multi-tool) is commonly carried by professionals in many industries, as well as casual and recreational users. The classic multi-function tool includes pliers or a pliers-like device that unfolds, unhinges, or slides out from within a pair of handles. The handles typically also serve as covers for additional tools, such as knives, screwdrivers, saws, bottle openers, etc., that fold out from within the handle by rotating on a pin or hinge located at the end opposite the main tool. As the products have matured, multi-function tools are also offered with tool attachments located along the sides and backs of the handles, and features such as cartridge-based tool tips have increased the number of useful tools that can be combined into one multi-function tool. In addition, handle shapes and textures have changed and become more stylish in the manufacturers' attempt to differentiate their products.

To date, however, no multi-function tool has addressed the needs of healthcare professionals with tools specific to their needs. These tools may include pill slicers for splitting large pills into the proper dosage, a practice that has become more common as medical facilities tend to buy common medication in standard sizes for cost savings. Valve wrenches are needed for operating medical gas cylinder valves, as it is necessary to open and close the valve during periods of oxygen administration.

Tools that are a little more common, but still used regularly by medical professionals, include small tape measures or folding rulers which are used when examining patients. Nurses and other healthcare providers carry small pliers with properly shaped jaws for actuating clamps on I.V. tubes. Penlights, or small flashlights, are used when examining patients. Tweezers or clippers are also used in several ways by examining nurses and other healthcare providers.

In some cases, specialists will carry additional tools specific to their duties. An example is a sharp/dull test kit, used by many to perform neurologic assessments. Similarly, calipers are often used on cardiac or ICU wards for measuring EKG intervals.

With the number of tasks and varied tools required to perform those tasks, healthcare professionals must carry enough tools to insure they have the proper tool for the task at hand. Nursing staff, for example, often work with small tool packs on their waist, in which they carry tools they use throughout the day. Others simply carry multiple tools in various pockets, or choose not to carry any at all. Searching through pockets or pouches to locate the proper tool or walking to and from the supply room takes much unnecessary time. Furthermore, the more individual tools involved, the higher the risk that a needed tool will be mistakenly left behind following a previous use.

Healthcare providers must also maintain as sterile an environment as possible. All tools must be sterilized prior to coming in contact with patients, and this is done most commonly by alcohol swabs, though other methods can be used. Any tool must therefore be capable of being easily cleaned and sterilized following use. Smooth, metallic surfaces present one of the easiest surfaces to maintain and sterilize. A single tool can be sterilized more quickly than multiple tools, since the amount of handling is reduced. Additionally, if an alcohol bath or similar sterilization technique is used, then all individual tools built into the multi-function tool would be sterilized at one time.

Therefore, a need exists for a single tool that combines as many of these common healthcare tools as possible into one easily carried unit, such that the most commonly needed tool is always handy and within reach. In addition, by combining the tools into one multi-tool, the total weight and volume of tools that a professional must carry can be reduced significantly. This will help each professional perform his or her job more efficiently and effectively, as well as cut down on the mental fatigue and frustration of not having the correct tool immediately available. To the extent this helps the professional work more quickly and efficiently, the quality of healthcare will be increased, and the response time for activities that require special tools will decrease.

In addition to the tool itself, a carrier for the tool that includes other commonly used devices would add to the utility of the multi-function tool. This carrier might include a loop for holding and dispensing tape from a roll, as well as a key ring on a retractable cable for holding keys and/or access badges used throughout healthcare facilities. The carrier is equipped with either clips or belt loops so that it can be carried easily on the waist.

Various tools with some of the features described exist in the prior art. For example:

U.S. patent application Ser. No. 11/232,042 (Gillingham, 2006) describes a tool designed for emergency responders that includes an opening in the handle suitable for use on domestic natural gas shut-off valves outside the home.

Similarly, U.S. Pat. No. 5,542,139 (Boivin, 1996) describes a folding tool designed primarily for emergency use, with a seat belt cutter and a punch for breaking through fabric or glass. The handle includes a slot designed to fit a domestic natural gas shut-off valve.

U.S. Pat. No. 6,764,301 (Paleno, et al., 2004) shows a combination striker and gas cylinder wrench which would be useful for welders, construction crewmen, and others who regularly use a cutting torch.

In the medical professional arena, U.S. Pat. No. 5,115,571 (Macklin, 1992) describes a combination pen and caliper device for the medical community, so that the calipers used to measure charts, and the pen used to write down the measurement, are housed together.

U.S. Pat. No. 6,450,069 (Brahmbhatt, 2002) describes a multi-purpose wrench which fits numerous valves on gas cylinders, including oxygen bottles used for medical treatment.

International Patent Application PCT/US2007/002836 (Boslough, et al., 2008) describes a compact tool that houses multiple tools used in testing neurological responses. The product associated with this application is currently marketed as the Neuro-7 and is one of the few tools marketed specifically to medical professionals.

The foregoing examples show that there is a need for a compact multi-function tool, suitable for use by medical staff in a sterile environment, that combines individual tools that nurses, doctors, and other healthcare professionals use on a daily basis.

### SUMMARY OF THE INVENTION

A multi-tool according to the present invention comprises a hinged head having a first and second jaw, each jaw having a working portion and a tang. A handle is rotatably coupled to each tang. A medical gas cylinder valve wrench is integrated with at least one of the handles.

According to another aspect of the invention, the multi-tool may also include a pill splitting device integrated into one of the handles. The pill splitting device comprises a base formed by the handle, a hinged cover, and a cutting blade attached to the cover perpendicular to the plane of the base. When the cover is closed, the cutting edge of the blade is brought into near proximity the base.

A further aspect of the invention includes the addition of EKG calipers, rotatably coupled to one of the handles at the end opposite the head, wherein the calipers comprise two measuring legs hinged together at the end nearest the handle. Each leg further comprises a thin tip at the end opposite the hinge, the arrangement making the calipers suitable for transferring distances between points on a chart to an associated measurement scale.

### BRIEF DESCRIPTION OF THE DRAWINGS

FIG. 1 shows one view of a preferred embodiment of a compact multi-function tool, showing features that make it uniquely suited to the healthcare professional.

FIG. 2 shows another view of the multi-function tool of FIG. 1, shown in its folded or closed position.

FIGS. 3a through 3c show various designs of the multi-tool handle with an integrated gas cylinder valve operator.

FIG. 4 is a detail of one embodiment of an integrated pill slicer.

FIG. 5 shows a further embodiment wherein the multi-function tool includes a pair of fold-out EKG calipers.

FIGS. 6a and 6b show alternate details for the EKG caliper mounts.

FIGS. 7a and 7b show alternate details for a sharp/dull test tool integrated into one of the handles.

### DETAILED DESCRIPTION OF EMBODIMENTS OF THE INVENTION

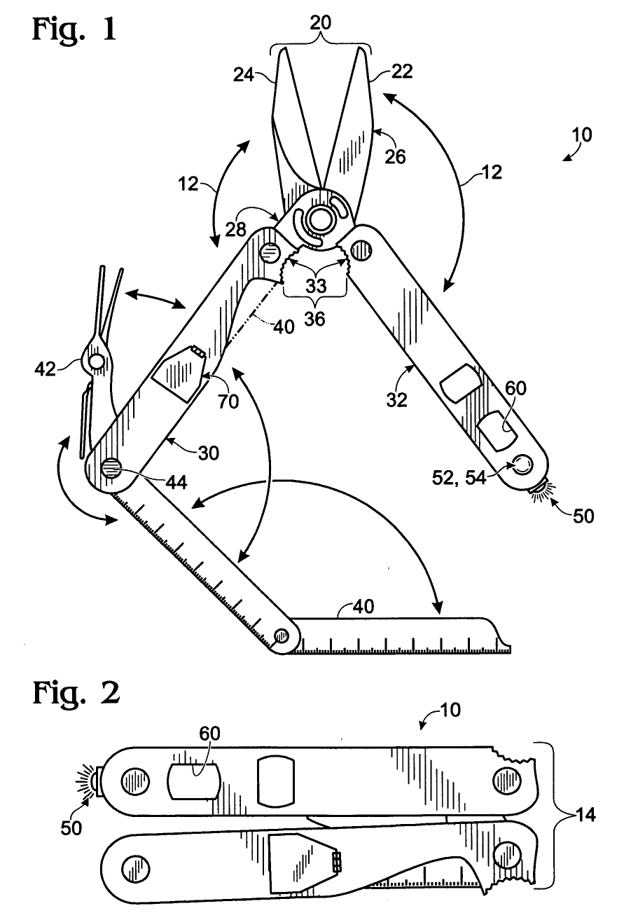

A preferred embodiment of the multi-function tool 10 according to the invention shown in FIG. 1 comprises hinged head 20 made of two jaws 22, 24. Each jaw comprises a working end 26, and a tang 28. The working end 26 shown in this illustration is shaped to act as scissors, though other shapes are commonly known to those skilled in the art, including pliers, wire cutters, wire strippers, and combination heads. Handles 30, 32 are rotatably coupled to each tang 28. The handles fold over the working end of the jaws, as shown by the arrows 12 in FIG. 1, to form a compact shape 14 as shown in FIG. 2. The mechanism for this feature is commonly known throughout the tool industry, and is not the subject of this invention.

As shown in FIG. 1, within handle 30, hinged ruler 40 and tweezers 42 are mounted on a hinge pin 44 located at the handle end opposite the tang. These tools would typically be used when the multi-function tool is in its folded position, though they would be accessible even when the multi-function tool is in the open position.

In a preferred embodiment as shown in FIG. 1, handles 30, 32 are equipped with properly shaped inside faces near the tang, or grip areas 33. When the handles are brought together so as to actuate the jaws' working ends 26, the grip areas form an I.V. cap grip 36. This cap grip is sized to firmly hold the industry-standard 1 centimeter diameter I.V. caps without crushing them.

Additionally, in the preferred embodiment shown in FIG. 1, handle 32 includes a small penlight 50 integrated into the handle. In this embodiment, penlight 50 is actuated by pushbutton 52. A button-shaped battery 54 is located under the body of pushbutton 52 and can be accessed by unscrewing the pushbutton from the handle 32. In the preferred embodiment, the light source is an LED bulb.

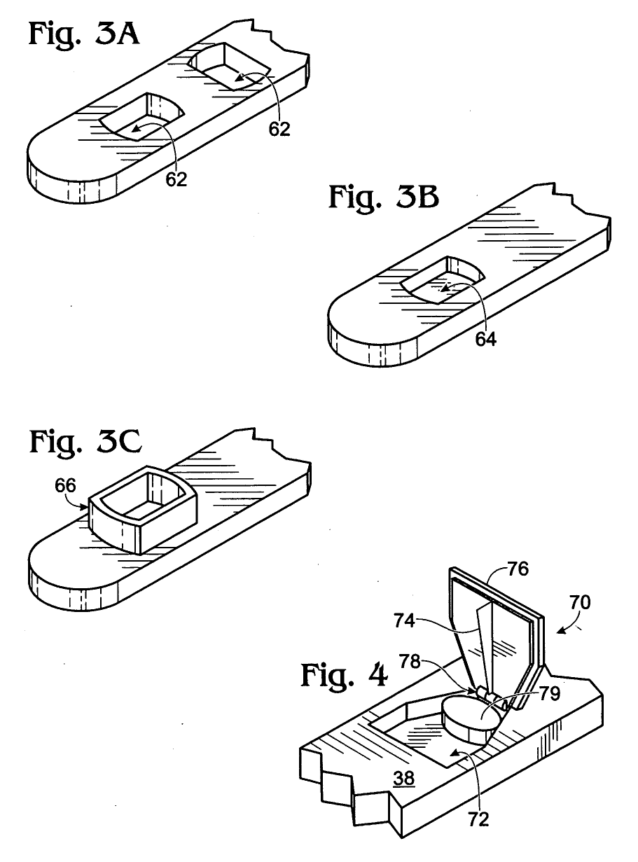

Handle 32 is also equipped with a properly shaped feature to form a gas cylinder wrench 60 integral to said handle, as shown in both FIG. 1 and FIG. 2. Turning to FIGS. 3a through 3c, the portion of the handle that engages a gas cylinder can be properly shaped through the use of a through hole 62 as shown in FIG. 3a; a depression or indentation 64 as shown in FIG. 3b; or a protrusion of walls 66 as shown in FIG. 3c. The protruding walls 66 of FIG. 3c may be formed by the handle itself, or by attaching separate material to the handle. The integral valve wrench 60 may be used when the multi-function tool is in the unfolded position as in FIG. 1 or the folded position as in FIG. 2, or in an intermediate position.

In the preferred embodiment, handle 30 is equipped with an integrated pill slicer 70, as shown in FIGS. 1 and 2. FIG. 4 shows one preferred embodiment of the integrated pill slicer, wherein the base 72 of the pill slicer 70 is formed as part of the handle surface 38. Blade 74 is attached to cover 76, perpendicular to base 72. Cover 76 is attached to base 72 by hinge 78. In the closed position, the cutting edge of blade 74 touches or nearly touches base 72. In use, a pill 79 is placed in the pill slicer 70 while cover 76 is open. The operator then closes cover 76 and in the process, the pill is sliced.

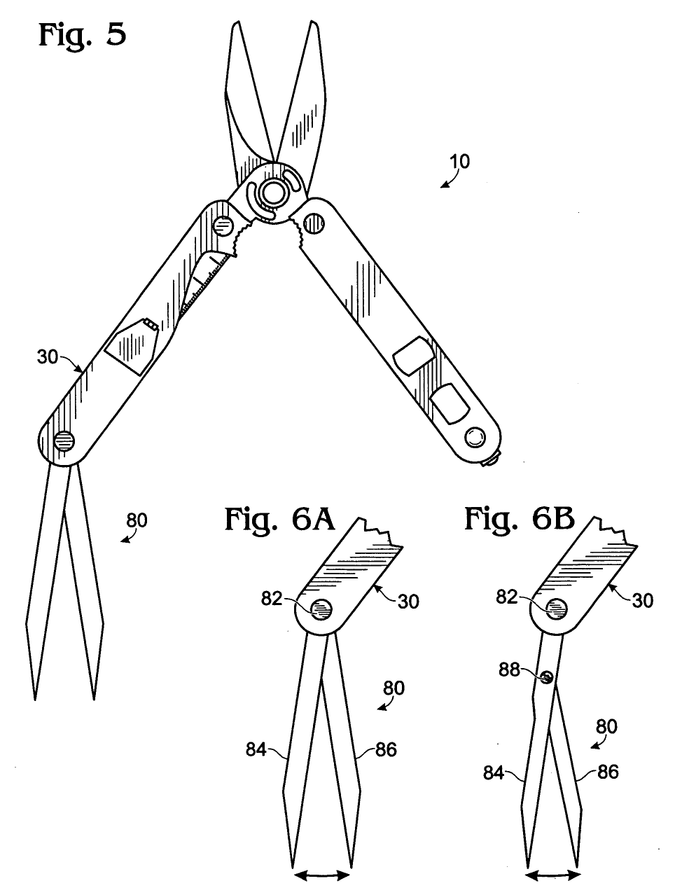

In a further embodiment, a multi-function tool handle 30 is equipped with a pair of EKG calipers 80, as shown in FIG. 5 and in more detail in FIG. 6a. The EKG calipers 80 are attached to the handle 30 via a hinge pin 82. In this embodiment, the hinge pin 82 used to mount the EKG calipers to the handle also acts as the functional hinge pin for the EKG calipers themselves, so that caliper legs 84, 86 can be split to measure distances on a chart. However, in another embodiment as shown in FIG. 6b, the EKG caliper's functional hinge pin 88 is separate from the mounting hinge pin 82.

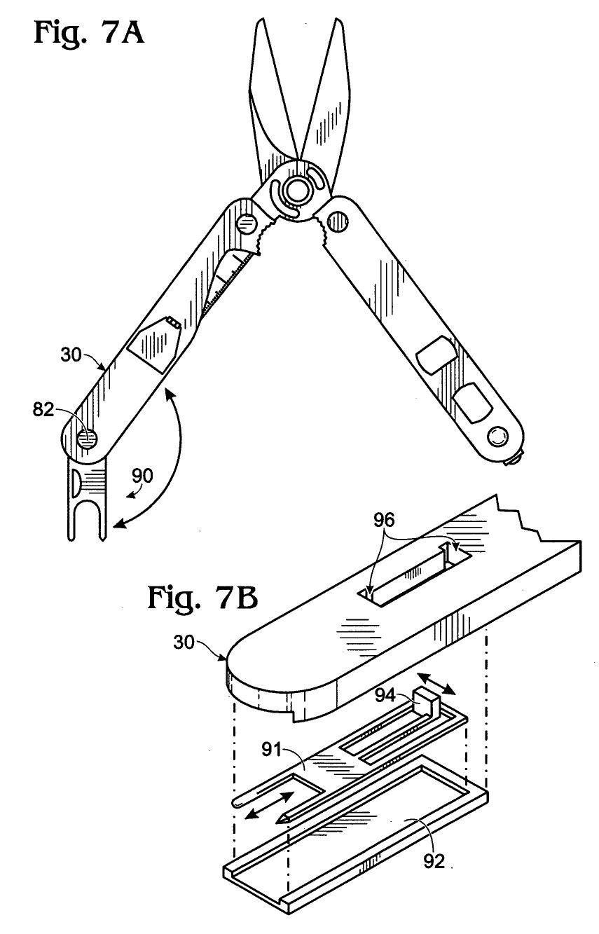

FIG. 7a shows handle 30 equipped with a sharp/dull test tool 90. In this embodiment, test tool 90 is rotatably attached to handle 30 by mounting hinge pin 82. In this embodiment, the test tool 90 rotates or folds into the cavity of handle 30. An alternative mounting method is shown in FIG. 7b, wherein test tool 91 is mounted within handle 30 on a slide tray 92. In this embodiment, the user moves the test tool to an extended or retracted position by pushing on the thumb tang 94. The tool is locked in the extended or retracted position when the thumb tang 94 is positioned at detents 96 in the handle. Thumb tang 94 is constructed so that the user must force the thumb tang out of the detent position by pushing sideways (to the right in this figure). Other locking devices common to the art may be used, including a vertical, sprung pushbutton that pops up in the fully retracted or extended position.

Each handle described above may be constructed of a single piece of metal, plastic, or other suitably rigid material. The handle may also, however, consist of one or more structural elements covered by a separate, exterior surfacing that is shaped appropriately to form the integral tools.

Other embodiments of the multi-function tool may not include all the features described above, or they may include additional tools. Such tools might include but not be limited to knife blades, saw blades, screwdrivers, bottle or can openers, punches, and awls, attached to the handle through the use of a hinge pin, other rotating coupling, or within cavities from which the tools slide out, separate from the multi-function tool. In another embodiment, the penlight would be replaced by a low-power diode laser pointer commonly available. While the figures show a tool in which the handles are attached to the jaw tangs via rotating couplings, such as hinge pins, the jaw tangs may also be coupled to the handles in such as manner as to allow the jaws to extend into working position by sliding the tang-to-handle connection point along the handle.

It is to be recognized that, while a particular multi-function tool has been shown as preferred, other configurations and methods could be utilized, in addition to those already mentioned, without departing from the principles of the invention.

The terms and expressions which have been employed in the foregoing specification are used therein as terms of description and not of limitation, and there is no intention in the use of such terms and expressions to exclude equivalents of the features shown and described or portions thereof, it being recognized that the scope of the invention is defined and limited only by the claims which follow.

1. A multi-function tool suitable for use by healthcare professionals, comprising:
a hinged head, said head comprising a first jaw and a second jaw, the first jaw having a working portion and a tang and the second jaw having a working portion and a tang;
first and second handles rotatably coupled to the respective jaw tangs; and
a gas cylinder valve wrench integral to one of said handles.

2. The multi-function tool of claim 1, wherein said integrated valve wrench comprises at least one through hole in said handle shaped to engage and operate the valve stem of a medical gas cylinder.

3. The multi-function tool of claim 2, further comprising:
a pill splitting device integral to one of said handles, said device comprising a base formed by said handle, a cover plate attached to one edge of said base by a hinge, such that said cover plate rotates towards said base when closing, and a cutting blade attached to said cover and arranged perpendicular to plane of said base, such that closing said cover brings the cutting edge of said blade into proximity with said base.

4. The multi-function tool of claim 3, further comprising:
a set of EKG calipers rotatably coupled to one of said handles at the end opposite the head, wherein said calipers comprise two measuring legs hinged together at the end nearest said handle, each measuring leg comprising a thin tip at the end opposite the hinge, said calipers suitable for transferring distances between points on a chart to an associated measurement scale.

5. The multi-function tool of claim 2, further comprising:
a set of EKG calipers rotatably coupled to one of said handles at the end opposite the head, wherein said dividers comprise two measuring legs hinged together at the end nearest said handle, each measuring leg comprising a thin tip at the end opposite the hinge, said calipers suitable for transferring distances between points on a chart to an associated measurement scale.

6. The multi-function tool of claim 1, wherein said integrated valve wrench comprises an elongate depression in the outer surface of said first handle, said depression shaped to engage and operate the stem of a shut-off valve of a medical gas cylinder.

7. The multi-function tool of claim 6, further comprising:
a pill splitting device integral to one of said handles, said device comprising a base formed by said handle, a cover plate attached to one edge of said base by a hinge, such that said cover plate rotates towards said base when closing, and a cutting blade attached to said cover and arranged perpendicular to plane of said base, such that closing said cover brings the cutting edge of said blade into proximity with said base.

8. The multi-function tool of claim 7, further comprising:
a set of EKG calipers rotatably coupled to one of said handles at the end opposite the head, wherein said dividers comprise two measuring legs hinged together at the end nearest said handle, each measuring leg comprising a thin tip at the end opposite the hinge, said calipers suitable for transferring distances between points on a chart to an associated measurement scale.

9. The multi-function tool of claim 6, further comprising:
a set of EKG calipers rotatably coupled to one of said handles at the end opposite the head, wherein said calipers consist of two measuring legs hinged together at the end nearest said handle, each measuring leg comprising a thin tip at the end opposite the hinge, said calipers suitable for transferring distances between points on a chart to an associated measurement scale.

10. The multi-function tool of claim 1, wherein said integrated valve wrench comprises elongate walls protruding from the surface of said handle, said walls configured to engage and operate the handle of a shut-off valve of a medical gas cylinder.

11. The multi-function tool of claim 10, further comprising:
a pill splitting device integral to one of said handles, said device comprising a base formed by said handle, a cover plate attached to one edge of said base by a hinge, such that said cover plate rotates towards said base when closing, and a cutting blade attached to said cover and arranged perpendicular to plane of said base, such that closing said cover brings the cutting edge of said blade into proximity with said base.

12. The multi-function tool of claim 11, further comprising:
A set of EKG calipers rotatably coupled to one of said handles at the end opposite the head, wherein said calipers comprise two measuring legs hinged together at the end nearest said handle, each measuring leg comprising a thin tip at the end opposite the hinge, said calipers suitable for transferring distances between points on a chart to an associated measurement scale.

13. The multi-function tool of claim 10, further comprising:
a set of EKG calipers rotatably coupled to one of said handles at the end opposite the head, wherein said calipers comprise two measuring legs hinged together at the end nearest said handle, each measuring leg comprising a thin tip at the end opposite the hinge, said dividers suitable for transferring distances between points on a chart to an associated measurement scale.

14. The multi-function tool of claim 1, further comprising:
an I.V. cap grip, wherein said cap grip comprises a grip area on the inside face of each said handle, said inside face defined as that portion of each handle which faces the opposite handle when the working portion of the jaws are in use, said grip area located such that when the handles are brought to the extreme closed position when actuating the working portion of the jaws, the grip areas form two sides of an opening that would allow the free passage of a sphere with diameter larger than 0.8 mm but prevent the passage of a sphere with diameter 1.0 cm or larger.

15. A method of operating a gas cylinder valve, comprising:
positioning a valve wrench to engage the operating stem of said cylinder valve, wherein said valve wrench is an integrated component of a first handle of a multi-function tool, said multi-function tool comprising a hinged head, said head comprising a first jaw and a second jaw, the first jaw having a working portion and a tang and the second jaw having a working portion and a tang, and said multi-function tool further comprising first and second handles rotatably coupled to the respective jaw tangs.
